(19) United States
(12) Patent Application Publication
(10) Pub. No.: US 2022/0273796 A1
(43) Pub. Date: Sep. 1, 2022

(54) LYOPHILIZED ANTIBODY FORMULATION

(71) Applicant: ICHNOS SCIENCES SA, La Chaux-de-Fonds (CH)

(72) Inventors: Sachin DUBEY, La Chaux-de-Fonds (CH); Brice GASSIAT, La Chaux-de-Fonds (CH)

(21) Appl. No.: 17/628,189

(22) PCT Filed: Jul. 16, 2020
(86) PCT No.: PCT/EP2020/070137

§ 371 Date: Jan. 18, 2022

(30) Foreign Application Priority Data
Jul. 19, 2019 (EP) 19187367.8

(51) Int. Cl. A61K 39/395; C07K 16/28; A61K 9/19; A61K 47/22; A61K 47/26

(52) U.S. Cl. CPC A61K 39/39591; C07K 16/2878; A61K 9/19; A61K 47/22; A61K 47/26; C07K 2317/76

(57) ABSTRACT

The present invention relates to a stable pharmaceutical formulation comprising an antibody; preferably the pharmaceutical formulation is lyophilized.

### TECHNICAL FIELD

The present invention relates to a stable pharmaceutical formulation comprising an antibody; preferably the pharmaceutical formulation is lyophilized.

### BACKGROUND

In the context of pharmaceutical product development, a pharmaceutical formulation refers to a mixture of an active pharmaceutical ingredient(s) and inactive ingredients, also known as excipients that are combined to produce a final drug product.

While the active pharmaceutical ingredient (API) has a pharmacological activity that contributes to the diagnosis, treatment, or prevention of a certain disease(s), the inactive ingredients have the role of contributing to product stability, biopharmaceutical profile, appearance and to patient acceptability (P. Furrer, European Pharmaceutical Review, Issue 2, 2013, Apr. 18, 2013). In particular the excipients, according to the International Pharmaceutical Excipients Council (IPEC, 1995) “are all substances contained in a dosage form other than the active substance or finished dosage form, which have been appropriately evaluated for safety and are included in a drug delivery system to either aid the processing of the drug delivery system during its manufacture, protect, support, enhance stability, bioavailability, or patient acceptability, assist in product identification, or enhance any other attributes of the overall safety and effectiveness of the drug delivery system during storage or use”. Excipients therefore include solvents, diluents, buffering agents, pH-adjusting agents, surfactants, preservatives, tonicity agents, stabilizing agents etc.

When developing a drug product, the choice of the pharmaceutical formulation (API(s) concentration, excipients, excipients concentration) depends on the properties of the API(s), on the drug product presentation, on the route of administration of the drug product and on the administration dosage. Among the factors driving such choice, there is the stability of the final drug product, for which integrity of the API and safety of the formulation for the patients must be assured.

For the production of therapeutic antibodies, the development of stable pharmaceutical formulations is necessary to obtain an effective and stable drug products, therefore optimizing antibody pharmaceutical formulations, and in parallel performing exhaustive stability tests, is a primary need of the pharmaceutical industry.

### SUMMARY

The present invention relates to a stable pharmaceutical formulation comprising an anti-OX40 antagonist antibody or antibody fragment thereof, one or more stabilizing or tonicity agent and a buffer, wherein said pharmaceutical formulation is liquid or lyophilized.

In one aspect, the pharmaceutical formulation of the present invention comprises a buffer selected from the group comprising acetate, L-histidine, citrate and phosphate and present within said pharmaceutical formulation at concentration between about 1 mM and about 10 mM.

In another aspect, the pharmaceutical formulation of the present invention comprises one or more stabilizing or tonicity agent selected from the group comprising sodium acetate, sodium bicarbonate, sodium carbonate, sodium chloride, potassium acetate, potassium bicarbonate, potassium carbonate, potassium chloride, calcium chloride, sucrose, glutamate, mannitol, polyols, Polysorbate 20, Polysorbate 40, Polysorbate 80, Poloxamer, Poloxamer 188, Poloxamer 407, amino acids such as histidine, arginine, glycine, methionine, proline, lysine, glutamic acid, amines, cyclodextrins, β-cyclodextrins, polyvinylpyrrolidone, polyethylene glycol 400, sorbitol, trehalose and EDTA, and present within said pharmaceutical formulation at a percentage between about 0.001% and about 10%.

In a particular embodiment, the pharmaceutical formulation of the present invention comprises an anti-OX40 antagonist antibody or fragment thereof present within said pharmaceutical formulation at a concentration between about 20 mg/mL and about 80 mg/mL, L-histidine buffer present within said pharmaceutical formulation at a concentration between about 1 mM and 10 mM, sucrose present within said pharmaceutical formulation at a percentage between about 1% and about 10% and Polysorbate 80 present within said pharmaceutical formulation at a percentage between about 0.001% and about 0.1%, and said pharmaceutical formulation has pH between about 5.0 and about 7.0.

In another particular embodiment, the pharmaceutical formulation of the present invention comprises an anti-OX40 antagonist antibody or fragment thereof present within said pharmaceutical formulation at a concentration between about 40 mg/mL and about 60 mg/mL, L-histidine buffer present within said pharmaceutical formulation at a concentration between about 3 mM and 7 mM, sucrose present within said pharmaceutical formulation at a percentage between about 2% and about 6% and Polysorbate 80 present within said pharmaceutical formulation at a percentage between about 0.005% and about 0.05%, and said pharmaceutical formulation has pH between about 5.5 and about 6.5.

In a preferred embodiment, the pharmaceutical formulation of the present invention comprises an anti-OX40 antagonist antibody or fragment thereof present within said pharmaceutical formulation at a concentration of about 48 mg/m L, L-histidine buffer present within said pharmaceutical formulation at a concentration of about 5 mM, sucrose present within said pharmaceutical formulation at a percentage of about 4% and Polysorbate 80 present within said pharmaceutical formulation at a percentage of about 0.01%, and said pharmaceutical formulation has pH of about 6.0.

According to one aspect of the present invention, the pharmaceutical formulation disclosed herein is lyophilized and it is stable at about +5±3° C. for at least 24 months, preferably for at least 48 moths, at about +25±2° C. for at least 12 months, and at about +40±2° C. for at least 3 months.

According to another aspect of the present invention, the lyophilized formulation is reconstituted and comprises an anti-OX40 antagonist antibody or antibody fragment thereof at a concentration comprised between about 0.5 mg/mL and bout 200 mg/m L.

According to a further aspect of the present invention, the lyophilized formulation is reconstituted with water for injection to obtain a reconstituted formulation comprising an anti-OX40 antagonist antibody or antibody fragment thereof at a concentration comprised between about 100 mg/mL and bout 200 mg/mL.

In certain embodiments, the reconstituted formulation is further diluted with a dilution media selected from the group comprising saline solution, glucose solution, dextrose solution, to obtain a diluted formulation comprising an anti-OX40 antagonist antibody or antibody fragment thereof at a concentration comprised between about 0.5 mg/mL and bout 5 mg/m L.

According to certain aspects of the present invention, the pharmaceutical formulation disclosed herein is suitable for subcutaneous administration or for intravenous administration.

The present invention also relates to pharmaceutical formulation as disclosed herein for use in the treatment of an OX40-mediated disorder.

In a specific aspect, the OX40-mediated disorder is selected from the group comprising atopic dermatitis, rheumatoid arthritis, autoimmune uveitis, multiple sclerosis, lupus (such as systemic lupus erythematosus), ulcerative colitis, scleroderma and graft-versus-host disease (GVHD), scleroderma, hidradenitis, and ulcerative colitis.

The present invention also disclose a method of manufacturing said pharmaceutical formulation.

The present invention also disclose an article of manufacture comprising the pharmaceutical formulation.

As used herein, the following terms have the following meanings: “a”, “an”, and “the” as used herein refers to both singular and plural unless the context clearly dictates otherwise.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of cell and tissue culture, molecular biology, and protein and oligo- or polynucleotide chemistry, laboratory procedures and techniques of analytical chemistry, synthetic organic chemistry, and medicinal and pharmaceutical chemistry described herein are those well-known and commonly used in the art.

Temperatures above 0° C. are expressed in the present invention either by preceding the temperature value by “+” or not, e.g. 25° C. and +25° C. can be used interchangeably according to the present invention.

The pharmaceutical formulation of the invention may be a liquid formulation, a lyophilized formulation or a reconstituted formulation.

A “liquid” formulation is one that has been prepared in a liquid format. Such a formulation may be suitable for direct administration to a subject or, alternatively, can be packaged for storage either in a liquid form, in a frozen state or in a dried form (e.g. lyophilized) for later reconstitution into a liquid form or other form suitable for administration to a subject.

A “lyophilized” formulation is one that has been prepared by freeze-drying a liquid or pre-lyophilization formulation. Freeze-drying is performed by freezing the formulation and then subliming ice from the frozen content at a temperature suitable for primary drying. Under this condition the product temperature is below the collapse temperature of the formulation. A secondary drying stage may then be carried out, which produces a suitable lyophilized cake.

A “reconstituted” formulation is one that has been prepared by dissolving a lyophilized protein formulation in a diluent, such as a reconstitution media, such that the protein is dispersed in the reconstituted formulation. The reconstituted formulation should be suitable for administration (e.g. parenteral administration) to a subject to be treated with the protein of interest. Additionally, the reconstituted formulation can be further “diluted” if a diluent is added to be ready for administration, for instance via an infusion system. Suitable diluent may be selected from the group comprising saline solution, dextrose solution, glucose solution and their combination.

Suitable “reconstitution media” useful for the preparation of a reconstituted formulation include ones which are pharmaceutically acceptable (safe and non-toxic for administration to a human). Examples of suitable diluents include sterile water for injection (SWFI), bacteriostatic water for injection (BWFI). Water for injection (WFI), a pH buffered solution e.g. phosphate-buffered saline (PBS), sterile saline solution, Ringer's solution or dextrose solution and the buffer used to prepare the pharmaceutical formulation.

The term “buffer” as used herein refers to a buffered solution that resists changes in pH by the action of its acid-base conjugate components. Examples of buffers that can control the pH in this range include acetate (e.g. sodium acetate), succinate (such as sodium succinate), gluconate, amino acids, such as histidine (e.g. histidine-HCl), citrate, phosphate, other organic acid buffer, their salts and combinations of buffers. A buffer of this invention has a pH in the range from about 5.0 to about 7.0; preferably the buffer has a pH selected from the group comprising pH about 5, pH about 5.5, pH about 6, pH about 6.5 and pH about 7. Examples of buffers that can control the pH in this range include acetate (e.g. sodium acetate), succinate (such as sodium succinate), gluconate, amino acids, such as histidine (e.g. L-histidine), citrate, phosphate, other organic acid buffer, their salts and combinations of buffers. In one embodiment of the present invention the buffer is present within the pharmaceutical formulation at concentration between about 0.1 mg/mL and about 2 mg/mL; preferably the buffer is present within the pharmaceutical formulation at concentration selected from the group comprising 0.1 mg/mL, 0.2 mg/mL, 0.4 mg/mL, 0.6 mg/mL, 0.8 mg/mL, 1 mg/mL, 1.2 mg/mL, 1.4 mg/mL, 1.6 mg/mL, 1.8 mg/mL and 2 mg/mL. The present invention also includes a buffer with a concentration at any intermediate value of the above said values. In a particular embodiment of the present invention, the buffer is L-Histidine, present within the pharmaceutical formulation at a concentration of about 0.8 mg/mL. In one embodiment of the present invention the buffer is present within the pharmaceutical formulation at concentration between about 0.1 mM and about 50 mM; preferably the buffer is present within the pharmaceutical formulation at concentration selected from the group comprising 0.1 mM, 0.5 mM, 1 mM, 5 mM, 10 mM, 15 mM, 20 mM, 25 mM, 30 mM, 35 mM, 40 mM, 45 mM and 50 mM. The present invention also includes a buffer with a concentration at any intermediate value of the above said values. In a particular embodiment of the present invention, the buffer is L-Histidine, present within the pharmaceutical formulation at a concentration of about 5 mM.

Preferably, an excipient is added to the formulation. Such excipients may typically include one or more surfactant, lyoprotectant, bulking agent, inorganic or organic salt, stabilizer, diluent, solubilizer, reducing agent, antioxidant, chelating agent, preservative, stabilizing or tonicity agents and the like.

Examples of stabilizing or tonicity agent include comprising sodium acetate, sodium bicarbonate, sodium carbonate, sodium chloride (NaCl), potassium acetate, potassium bicarbonate, potassium carbonate, potassium chloride, calcium chloride (CaCl2)) sugars such as sucrose, glucose and trehalose, polyols such as mannitol, maltitol, sorbitol, xylitol, erythritol, and isomalt, polyethylene glycol, such as PEG400, Ethylenediaminetetraacetic acid (EDTA), amino acids such as histidine (e.g. histidine-HCl), arginine (e.g. arginine hydrochloride) and glycine, methionine, proline, lysine (e.g. lysine-HCl), glutamic acid, glutamine, cysteine, amines, glutathione, cyclodextrin, such as such as Hydroxypropyl β-cyclodextrin (HPBCD), Hydroxypropyl-sulfobutyl β-cyclodextrin (HPSBCD), Sulfobutylether β-cyclodextrin (SBECD), β-cyclodextrin (BetaCD), α-cyclodextrin (Alpha CD) and γ-cyclodextrin (Gamm CD) and surfactants. Non limiting examples of a typical surfactant include: non-ionic surfactants (HLB 6 to 18) such as sorbitan fatty acid esters (e.g. sorbitan monocaprylate, sorbitan monolaurate, sorbitan monopalmitate), glycerine fatty acid esters (e.g. glycerine monocaprylate, glycerine monomyristate, glycerine monostearate), poly glycerine fatty acid esters (e.g. decaglyceryl monostearate, decaglyceryl distearate, decaglyceryl monolinoleate), polyoxyethylene sorbitan fatty acid esters (e.g. polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g. polyoxyethylene sorbitol tetrastearate, polyoxyethylene sorbitol tetraoleate), polyoxyethylene glycerine fatty acid esters (e.g. polyoxyethylene glyceryl monostearate), polyethylene glycol fatty acid esters (e.g. polyethylene glycol distearate), polyoxyethylene alkyl ethers (e.g. polyoxyethylene lauryl ether), polyoxy ethylene polyoxypropylene alkyl ethers (e.g. polyoxyethylene polyoxypropylene glycol ether, polyoxyethylene polyoxypropylene propyl ether, polyoxyethylene polyoxypropylene cetyl ether), polyoxyethylene alkylphenyl ethers (e.g. polyoxyethylene nonylphenyl ether), polyoxyethylene hydrogenated castor oils (e.g. polyoxyethylene castor oil, polyoxyethylene hydrogenated castor oil), polyoxyethylene beeswax derivatives (e.g. polyoxyethylene sorbitol beeswax), polyoxyethylene lanolin derivatives (e.g. polyoxyethylene lanolin), and polyoxyethylene fatty acid amides (e.g. polyoxyethylene stearyl amide); anionic surfactants such as Cio-Cis alkyl sulfates salts (e.g. sodium cetyl sulfate, sodium lauryl sulfate, sodium oleyl sulfate), polyoxyethylene Cio-Cis alkyl ether sulfates salts with an average of 2-4 moles of ethylene oxide (e.g. sodium polyoxyethylene lauryl sulfate), and Cs-Cis alkyl sulfosuccmate ester salts (e.g. sodium lauryl sulfosuccmate ester); natural surfactants such as lecithin, glycerophospho lipid, sphingophospho lipids (e.g. sphingomyelin) and sucrose esters of C 12-C 18 fatty acids; Poloxamers such as Poloxamer 188, Poloxamer 407, Poloxamer 124, Poloxamer 237, Poloxamer 338; salts and combinations of the above cited components. Preferably, the surfactant is selected from polyoxyethylene sorbitan fatty acid esters. Particularly preferably the surfactant is Polysorbate 20, 21, 40, 60, 65, 80, 81 and 85, most preferably Polysorbate 80. Polysorbate 80 is also known by the brand name Tween 80™ (ICI Americas, Inc.).

Examples of solubilizers include amino acids such as proline or glycine, prophylene glycol, plasdone C and K povidones, cyclodextrins and plasdone K polymers. Example of reducing agents, antioxidants and/or chelating agents. Examples of a reducing agent include N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione and a C1-C7 thioalkanoic acid. Examples of an antioxidant include amino acids such as methionine, erythorbic acid, dibutylhydroxytoluene, butylhydroxyanisole, alpha-tocopherol, tocopherol acetate, L-ascorbic acid and a salt thereof, L-ascorbic acid palmitate, L-ascorbic acid stearate, sodium bisulfite, sodium sulfite, triamyl gallate and propyl gallate. Examples of a chelating agent include disodium ethylenediammetetraacetate (EDTA), sodium pyrophosphate and sodium metaphosphate.

In certain embodiments of the present invention, the stable pharmaceutical formulation comprise one or more stabilizing or tonicity agent at a concentration between about 0.05 mg/mL and about 50 mg/mL, preferably at a concentration selected from the group comprising about 0.05 mg/mL, about 0.1 mg/mL, about 0.5 mg/mL, about 1 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/m L. The present invention also includes one or more stabilizing or tonicity agent with a concentration at any intermediate value of the above said values. In a particular embodiment of the present invention, the one or more stabilizing or tonicity agents are sucrose present within the pharmaceutical formulation at a concentration of about 40 mg/mL and Polysorbate 80 present within the pharmaceutical formulation at a concentration of about 0.1 mg/mL. In certain embodiments of the present invention, the stable pharmaceutical formulation comprise one or more stabilizing or tonicity agent at a percentage between about 0.001% and about 10%, preferably at a concentration selected from the group comprising about 0.001%, about 0.005%, about 0.01%, about 0.05%, about 0.1%, about 0.5%, about 1%, about 2%, about 4%, about 5%, about 8%, about 10%. The present invention also includes one or more stabilizing or tonicity agent with a percentage at any intermediate value of the above said values. In a particular embodiment of the present invention, the one or more stabilizing or tonicity agents are sucrose present within the pharmaceutical formulation at a percentage of about 4% and Polysorbate 80 present within the pharmaceutical formulation at a percentage of about 0.01%.

Not all excipients in a liquid formulation are suitable for inclusion a lyophilized form of the formulation and therefore a number of changes may be made to the liquid formulation to make it suitable for freeze-drying.

A stabilizer may be added to the formulation to stabilise the protein in the lyophilised form. Examples of a stabiliser include creatinine, an amino acid selected from histidine, alanine, glutamic acid, glycine, leucine, phenylalanine, methionine, isoleucine, proline, aspartic acid, arginine, lysine and threonine, a carbohydrate selected from sucrose, trehalose, sorbitol, xylitol and mannose, surfactants selected from polyethylene glycol (PEG; e.g. PEG3350 or PEG4000) or polyoxyethylene sorbitan fatty acid esters (e.g. Polysorbate 20 or Polysorbate 80), or any combination thereof. In a preferred embodiment the stabiliser comprises a lyoprotectant, which may be selected from a non-reducing sugar (e.g. sucrose or trehalose) and/or a sugar alcohol (e.g. mannitol). Addition of a lyoprotectant helps to reduce the amount of degradation or aggregation of the protein upon lyophilisation. For instance, the lyophilized formulation is isotonic upon reconstitution; therefore the amount of lyoprotectant in the lyophilized formulation should be sufficient to achieve an isotonic reconstituted formulation. Alternatively, the reconstituted formulation may be hypertonic and therefore a greater amount of lyoprotectant is required in the lyophilized formulation.

A preservative may be added to the diluent to reduce bacterial action in the reconstituted formulation Examples of a preservative include octadecyldimethylbenzyl ammonium chloride, hexamethonium chloride, benzalkonium chloride (a mixture of alkylbenzyldimethyl-ammonium chlorides in which the alkyl groups are long-chain compounds), benzethonium chloride, aromatic alcohols such as phenol, butyl and benzyl alcohol, alkyl parabens such as methyl or propyl paraben, catechol, resorcinol, cyclohexanol, 3-pentanol, and m-cresol.

The lyophilized formulation may be reconstituted with a diluent. Diluents for use in reconstitution of the lyophilized formulation include sterile water, bacteriostatic water for injection (BWFI), water for injection (WFI) a pH buffered solution such as phosphate buffered saline, sterile saline solution, Ringer's solution or dextrose solution.

The pharmaceutical formulation according to the present invention also comprises an anti-OX40 antagonist antibody or an antibody fragment thereof.

The term “antibody” as referred to herein includes whole antibodies and any antigen binding fragments or single chains thereof. An “antibody” refers to a glycoprotein comprising at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR) with are hypervariable in sequence and/or involved in antigen recognition and/or usually form structurally defined loops, interspersed with regions that are more conserved, termed framework regions (FR or FW). Each VH and VL is composed of three CDRs and four FWs, arranged from amino-terminus to carboxy-terminus in the following order: FW1, CDR1, FW2, CDR2, FW3, CDR3, FW4. The amino acid sequences of FW1, FW2, FW3, and FW4 all together constitute the “non-CDR region” or “non-extended CDR region” of VH or VL as referred to herein.

The term “heavy chain variable framework region” as referred herein may comprise one or more (e.g., one, two, three and/or four) heavy chain framework region sequences (e.g., framework 1 (FW1), framework 2 (FW2), framework 3 (FW3) and/or framework 4 (FW4)). Preferably the heavy chain variable region framework comprises FW1, FW2 and/or FW3, more preferably FW1, FW2 and FW3. The term “light chain variable framework region” as referred herein may comprise one or more (e.g., one, two, three and/or four) light chain framework region sequences (e.g., framework 1 (FW1), framework 2 (FW2), framework 3 (FW3) and/or framework 4 (FW4)). Preferably the light chain variable region framework comprises FW1, FW2 and/or FW3, more preferably FW1, FW2 and FW3.

The variable regions of the heavy and light chains contain a binding domain that interacts with an antigen. The constant regions of the antibodies may mediate the binding of the immunoglobulin to host tissues or factors, including various cells of the immune system (e.g., effector cells) and the First component (C1q) of the classical complement system.

Antibodies are grouped into classes, also referred to as isotypes, as determined genetically by the constant region. Human constant light chains are classified as kappa (CK) and lambda (CX) light chains. Heavy chains are classified as mu (μ), delta (δ), gamma (γ), alpha (α), or epsilon (ε), and define the antibody's isotype as IgM, IgD, IgG, IgA, and IgE, respectively. Thus, “isotype” as used herein is meant any of the classes and/or subclasses of immunoglobulins defined by the chemical and antigenic characteristics of their constant regions. The known human immunoglobulin isotypes are IgG1 (IGHG1), IgG2 (IGHG2), IgG3 (IGHG3), IgG4 (IGHG4), IgA1 (IGHA1), IgA2 (IGHA2), IgM (IGHM), IgD (IGHD), and IgE (IGHE). The so-called human immunoglobulin pseudo-gamma IGHGP gene represents an additional human immunoglobulin heavy constant region gene which has been sequenced but does not encode a protein due to an altered switch region (Bensmana M et al., (1988) Nucleic Acids Res. 16(7): 3108). In spite of having an altered switch region, the human immunoglobulin pseudo-gamma IGHGP gene has open reading frames for all heavy constant domains (CH1-CH3) and hinge. All open reading frames for its heavy constant domains encode protein domains which align well with all human immunoglobulin constant domains with the predicted structural features. This additional pseudo-gamma isotype is referred herein as IgGP or IGHGP. Other pseudo immunoglobulin genes have been reported such as the human immunoglobulin heavy constant domain epsilon PI and P2 pseudo-genes (IGHEP1 and IGHEP2). The IgG class is the most commonly used for therapeutic purposes. In humans this class comprises subclasses IgG1, IgG2, IgG3 and IgG4. In mice this class comprises subclasses IgG1, IgG2a, IgG2b, IgG2c and IgG3.

The terms “antagonistic antibody” or “antagonist antibody” are used herein equivalently and include an antibody that is capable of inhibiting and/or neutralising the biological signaling activity of OX40, for example by blocking binding or substantially reducing binding of OX40 to OX40 ligand and thus inhibiting or reducing the signalisation pathway triggered by OX40 and/or inhibiting or reducing an OX40-mediated cell response like lymphocyte proliferation, cytokine expression, or lymphocyte survival.

The term “anti-OX40 antagonist antibody or fragment thereof” is used herein to indicate antibodies or antibody fragments thereof that bind to OX40 e.g. human OX40, and are capable of inhibiting and/or neutralising the biological signalling activity of OX40, for example by blocking binding or substantially reducing binding of OX40 to OX40 ligand and thus inhibiting or reducing the signalisation pathway triggered by OX40 and/or inhibiting or reducing an OX40-mediated cell response like lymphocyte proliferation, cytokine expression, or lymphocyte survival.

Antibody fragments include, but are not limited to, (i) the Fab fragment consisting of VL, VH, CL and CHI domains, including Fab′ and Fab′-SH, (ii) the Fd fragment consisting of the VH and CHI domains, (iii) the Fv fragment consisting of the VL and VH domains of a single antibody; (iv) the dAb fragment (Ward E S et al., (1989) Nature, 341: 544-546) which consists of a single variable, (v) F(ab′)2 fragments, a bivalent fragment comprising two linked Fab fragments (vi) single chain Fv molecules (scFv), wherein a VH domain and a VL domain are linked by a peptide linker which allows the two domains to associate to form an antigen binding site (Bird R E et al, (1988) Science 242: 423-426; Huston J S et al, (1988) Proc. Natl. Acad. Sci. USA, 85: 5879-83), (vii) bispecific single chain Fv dimers (PCT/US92/09965), (viii) “diabodies” or “triabodies”, multivalent or multispecific fragments constructed by gene fusion (Tomlinson I & Hollinger P (2000) Methods Enzymol. 326: 461-79; WO94/13804; Holliger P et al, (1993) Proc. Natl. Acad. Sci. USA, 90: 6444-48) and (ix) scFv genetically fused to the same or a different antibody (Coloma M J & Morrison S L (1997) Nature Biotechnology, 15(2): 159-163).

In a particular embodiment of the present invention, the stable pharmaceutical formulation comprises an antibody or antibody fragment thereof present within said pharmaceutical formulation at a concentration between about 5 mg/mL and about 100 mg/mL, preferably between about 20 mg/mL and about 80 mg/mL, more preferably between about 40 mg/mL and about 60 mg/mL. In particular, the stable pharmaceutical formulation of the present invention comprises an antibody or antibody fragment at a concentration selected from the group comprising about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 48 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL. The present invention also includes an antibody or antibody fragment thereof with a concentration at any intermediate value of the above said values. In a particular embodiment of the present invention, the antibody or antibody fragment thereof is present within the pharmaceutical formulation at a concentration of about 48 mg/m L.

In one aspect of the present invention the formulation according to the present invention comprises an anti-OX40 antagonist antibody or fragment thereof, a buffer and one or more stability or tonicity agents. In a particular aspect the pharmaceutical formulation comprises histidine buffer and/or sucrose and/or Polysorbate 80. In a more particular aspect the pharmaceutical formulation of the present invention comprises Histidine buffer at a concentration between about 0.1 mM and about 50 mM, preferably between about 1 mM and about 10 mM, more preferably at a concentration of about 5 mM; Polysorbate 80 at a concentration between about 0.0001% and 10%, preferably between about 0.005% and about 1%, preferably between about 0.001% and about 1%, more preferably at a concentration of about 0.01% and sucrose at a concentration between about 0.1% and 10%, preferably between about 1% and about 8%, more preferably at a concentration of about 4%. In a specific aspect of the present application, the anti-OX40 antagonist antibody or fragment thereof is present within the pharmaceutical formulation at a concentration between about 1 mg/mL and about 100 mg/mL, preferably at a concentration of about 50 mg/mL, more preferably at a concentration of about 48 mg/mL. The present invention also discloses concentrations of the anti-OX40 antagonist antibody or fragment thereof, of the buffer and of the stability or tonicity agents at any value intermediate to the above cited values. According to one aspect of the present invention the pharmaceutical formulation has a pH between about 5 and 7, preferably of about 6.

In a certain embodiment, the present invention discloses a pharmaceutical formulation wherein said anti-OX40 antagonist antibody or fragment thereof is present within said pharmaceutical formulation at a concentration between about 20 mg/mL and about 80 mg/mL, L-histidine buffer is present within said pharmaceutical formulation at a concentration between about 1 mM and 10 mM, sucrose is present within said pharmaceutical formulation at a percentage between about 1% and about 10% and Polysorbate 80 present within said pharmaceutical formulation at a percentage between about 0.001% and about 0.1%, and wherein said pharmaceutical formulation has pH between about 5.0 and about 7.0.

In a certain embodiment, the present invention discloses a pharmaceutical formulation wherein said anti-OX40 antagonist antibody or fragment thereof is present within said pharmaceutical formulation at a concentration between about 40 mg/mL and about 60 mg/mL, L-histidine buffer is present within said pharmaceutical formulation at a concentration between about 3 mM and 7 mM, sucrose is present within said pharmaceutical formulation at a percentage between about 2% and about 6% and Polysorbate 80 present within said pharmaceutical formulation at a percentage between about 0.005% and about 0.05%, and wherein said pharmaceutical formulation has pH between about 5.5 and about 6.5.

In one aspect the pharmaceutical formulation of the present invention is lyophilized. In one embodiment the lyophilizate comprises histidine at an amount between about 0.1 mg and about 50 mg, preferably between about 1 mg and about 10 mg, more preferably at an amount of about 4 mg or at an amount of about 3.1 mg, Polysorbate 80 at an amount between about 0.01 mg and 10 mg, preferably between about 0.1 mg and 1 mg, more preferably at an amount of about 0.5 mg or at an amount of about 0.4 mg and sucrose at an amount between about 100 mg and 300 mg, preferably between about 150 mg and about 250 mg, more preferably at an amount of about 200 mg or at an amount of about 160 mg. In a specific aspect of the present application, the anti-OX40 antagonist antibody or fragment thereof is present within the pharmaceutical formulation at an amount between about 100 mg and about 350 mg, preferably between about 150 mg and about 250 mg, more preferably at an amount of about 240 mg or at an amount of about 192 mg. The present invention also discloses concentrations of the anti-OX40 antagonist antibody or fragment thereof, of the buffer and of the stability or tonicity agents at any value intermediate to the above cited values.

According to one aspect of the present invention, a reconstitution medium such as water for injection (WFI) is added to the lyophilized formulation such that the antibody concentration in the reconstituted formulation is present in an amount of between about 0.1 mg/ml and about 250 mg/ml, preferably between about 0.5 mg/ml and about 200 mg/ml. In certain aspects of the present invention the antibody concentration in the reconstituted formulation between about 100 mg/ml and about 200 mg/ml, more preferably the antibody concentration in the reconstituted formulation is selected from the group comprising about 100 mg/ml, about 120 mg/mL, about 150 mg/mL, about 170 mg/mL, about 200 mg/mL; more preferably the antibody concentration in the reconstituted formulation is about 150 mg/mL. The present invention also includes antibody concentrations at any intermediate value of the above said values.

In other particular aspects of the present invention, the reconstituted formulation is further diluted with a dilution media selected from the group comprising saline solution, glucose solution, dextrose solution, and their combinations to obtain a diluted formulation comprising said antibody or antibody fragment thereof at a concentration below 200 mg/mL, or below 150 mg/mL, below 100 mg/mL, below 50 mg/mL, below 10 mg/mL, below 5 mg/mL; preferably comprised between about 0.1 mg/mL and bout 150 mg/mL; more preferably between about 0.5 mg/mL and bout 5 mg/mL. In certain aspects, the antibody concentration in the diluted formulation is selected from the group comprising about 0.5 mg/ml, about 1 mg/mL, about 1.2 mg/mL, about 1.5 mg/mL, about 1.8 mg/mL, about 2 mg/mL, about 2.2 mg/mL, about 2.5 mg/mL, about 2.8 mg/mL, about 3 mg/mL about 3.2 mg/mL, about 3.5 mg/mL, about 3.8 mg/mL, about 4 mg/mL about 4.2 mg/mL, about 4.5 mg/mL, about 4.8 mg/mL, about 5 mg/mL; more preferably the antibody concentration in the reconstituted formulation is about 2.2 mg/m L. The present invention also includes antibody concentrations at any intermediate value of the above said values.

The pharmaceutical formulation according to the present invention is stable. A “stable” formulation is one in which the protein therein essentially retains its physical stability and/or chemical stability and/or biological activity upon storage. Various analytical techniques for measuring protein stability are available in the art and are reviewed for example in Peptide and Protein Drug Delivery, 247-301, Vincent Lee Ed., Marcel Dekker, Inc., New York, N.Y., Pubs. (1991) and Jones A (1993) Adv Drug Delivery Rev, 10: 29-90. Stability can be measured at a selected temperature for a selected time period.

Analytical tests useful to determine said stability include but are not limited to: monitoring of the visual appearance as a significant change in the appearance of sample may indicate product degradation and/or microbial contamination leading to safety risk for the patients; sub-visible particles analysis, as the presence of higher sub-visible particles in parental solutions may lead to immunogenic responses; protein content measurement (e.g. by measuring absorbance at 280 nm wavelength (A280) by UV-VIS Spectroscopy or by SoloVPE) as any significant variation from its target concentration would not provide effective dose to patients; pH measurement as changes in pH may be indicative of degradation of buffering agents and lead to protein instability; size variants monitoring (e.g. by SE-HPLC and/or by cGE reduced and non-reduced) as changes in monomeric content toward aggregates (higher size than monomer) or fragments (smaller size than monomer) is an indication of its degradation; charge variants monitoring (e.g. by clEF) as changes in content of charged variants is an indication of its degradation; antibody potency measurement (e.g. by ELISA) as any significant change of binding property of the antibody toward its target would indicate antibody degradation. Additionally, the amino acid sequence as well as post-translational modifications (i.e. deamidation, oxidation, glycation, N-terminal variants, C-terminal variants and glycosylation site occupancy) can be verified, for instance by peptide mapping. Other characteristics of the formulation can be monitored, such as osmolarity and viscosity, as well as the protein thermal stability for instance by nano-format of Differential Scanning Fluorimetry (DSF).

In one aspect of the present invention, the pharmaceutical formulation is stable at about +5±3° C. for at least 48 months, at about +25±2° C. for at least 12 months, and at about +40±2° C. for at least 3 months.

In a particular embodiment, the anti-OX40 antibody or fragment thereof is a humanized monoclonal anti-OX40 antagonist antibody or fragment thereof.

The term “humanized antibody” or “humanized anti-OX40 antibody” as used herein includes antibodies in which CDR sequences derived from the germline of another mammalian species, such as a mouse, have been grafted onto human framework sequences. Additional framework region modifications may be made within the human framework sequences as well as within the CDR sequences derived from the germline of another mammalian species.

More specifically, the anti-OX40 antagonist antibody or fragment is GBR830 (CAS Registry Number 2126777-87-3).

In a certain embodiment, the pharmaceutical formulation of the present invention can be used in the treatment of patients suffering of an OX40-mediated disorders.

As used herein, the term “OX40-mediated disorder” includes conditions such as allergy, asthma, COPD, rheumatoid arthritis, psoriasis and diseases associated with autoimmunity and inflammation. In particular, according to the present invention, exemplary OX40 mediated disorders include infections (viral, bacterial, fungal and parasitic), endotoxic shock associated with infection, arthritis, rheumatoid arthritis, asthma, chronic obstructive pulmonary disease (COPD), pelvic inflammatory disease, Alzheimer's Disease, inflammatory bowel disease, Crohn's disease, ulcerative colitis, Peyronie's Disease, coeliac disease, gallbladder disease, Pilonidal disease, peritonitis, psoriasis, vasculitis, surgical adhesions, stroke, Type I Diabetes, lyme disease, arthritis, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, pancreatitis, trauma (surgery), graft-versus-host disease (GVHD), transplant rejection, cardiovascular disease including ischaemic diseases such as myocardial infarction as well as atherosclerosis, intravascular coagulation, bone resorption, osteoporosis, osteoarthritis, periodontitis, hypochlorhydia, hidradenitis and neuromyelitis optica.

Other exemplary OX40 mediated disorder include infections (viral, bacterial, fungal and parasitic), endotoxic shock associated with infection, arthritis, rheumatoid arthritis, asthma, bronchitis, influenza, respiratory syncytial virus, pneumonia, chronic obstructive pulmonary disease (COPD), idiopathic pulmonary fibrosis (IPF), cryptogenic fibrosing alveolitis (CFA), idiopathic fibrosing interstitial pneumonia, emphysema, pelvic inflammatory disease, Alzheimer's Disease, inflammatory bowel disease, Crohn's disease, ulcerative colitis, Peyronie's Disease, coeliac disease, gallbladder disease, Pilonidal disease, peritonitis, psoriasis, vasculitis, surgical adhesions, stroke, Type I Diabetes, lyme disease, arthritis, meningoencephalitis, autoimmune uveitis, immune mediated inflammatory disorders of the central and peripheral nervous system such as multiple sclerosis, lupus (such as systemic lupus erythematosus) and Guillain-Barr syndrome, Atopic dermatitis, autoimmune hepatitis, fibrosing alveolitis, Grave's disease, IgA nephropathy, idiopathic thrombocytopenic purpura, Meniere's disease, pemphigus, primary biliary cirrhosis, sarcoidosis, scleroderma, Wegener's granulomatosis, pancreatitis, trauma (surgery), graft-versus-host disease (GVHD), transplant rejection, cardiovascular disease including ischaemic diseases such as myocardial infarction as well as atherosclerosis, intravascular coagulation, bone resorption, osteoporosis, osteoarthritis, periodontitis, hypochlorhydia, hidradenitis and neuromyelitis optica.

In accordance to a preferred aspect of the present invention, the pharmaceutical formulation of the present invention is used for the treatment or prevention of an OX40-mediated disorder selected from the group comprising atopic dermatitis, rheumatoid arthritis, autoimmune uveitis, multiple sclerosis, lupus (such as systemic lupus erythematosus), ulcerative colitis, scleroderma and graft-versus-host disease (GVHD), scleroderma, hidradenitis, and ulcerative colitis.

The present invention provides a pharmaceutical formulation for use in the treatment of an OX40-mediated disorder selected from the group comprising atopic dermatitis, rheumatoid arthritis, autoimmune uveitis, multiple sclerosis, lupus (such as systemic lupus erythematosus), ulcerative colitis, scleroderma and graft-versus-host disease (GVHD), scleroderma, hidradenitis, and ulcerative colitis.

The present invention also provides a method for treating an OX40-mediated disorder using the pharmaceutical formulation of the present invention, wherein the OX40-mediated disorder is selected from the group comprising atopic dermatitis, rheumatoid arthritis, autoimmune uveitis, multiple sclerosis, lupus (such as systemic lupus erythematosus) and graft-versus-host disease (GVHD), scleroderma, hidradenitis, and ulcerative colitis.

As used herein, the term “subject” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc. Preferably the subject is human.

A “patient” for the purposes of the present invention includes both humans and other animals, preferably mammals and most preferably humans. Thus the antibodies of the present invention have both human therapy and veterinary applications. The term “treatment” or “treating” in the present invention is meant to include therapeutic treatment, as well as prophylactic, or suppressive measures for a disease or disorder. Thus, for example, successful administration of an antibody prior to onset of the disease results in treatment of the disease. As another example, successful administration of an antibody after clinical manifestation of the disease to combat the symptoms of the disease comprises treatment of the disease.

“Treatment” and “treating” also encompasses administration of an antibody after the appearance of the disease in order to eradicate the disease. Successful administration of an antibody after onset and after clinical symptoms have developed, with possible abatement of clinical symptoms and perhaps amelioration of the disease, comprises treatment of the disease. Those “in need of treatment” include mammals already having the disease or disorder, as well as those prone to having the disease or disorder, including those in which the disease or disorder is to be prevented.

The antibody or of the present invention can be administered via one or more routes of administration using one or more of a variety of methods known in the art. As will be appreciated by the skilled artisan, the route and/or mode of administration will vary depending upon the desired results. Preferred routes of administration include intravenous, intramuscular, intradermal, intraperitoneal, subcutaneous, spinal or other parenteral routes of administration, for example by injection or infusion. More preferred routes of administration are intravenous or subcutaneous. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the invention can be administered via a non-parenteral route, such as a topical, epidermal or mucosal route of administration, for example, intranasally, orally, vaginally, rectally, sublingually or topically.

According to a specific aspect of the present invention, the pharmaceutical formulation of the present invention is suitable for subcutaneous administration, or via intravenous infusion. In a particular aspect of the present invention, the pharmaceutical formulation of the present invention suitable for subcutaneous administration comprises the anti-OX40 antibody or fragment thereof at a concentration of about 150 mg/mL. In another particular aspect of the present invention, the pharmaceutical formulation of the present invention suitable for intravenous administration comprises the anti-OX40 antibody or fragment thereof at a concentration of about 2.2 mg/m L.

The antibody of the present invention can be administered at a single or multiple doses. The term “dose” or “dosage” as used in the present invention are interchangeable and indicates an amount of drug substance administered per body weight of a subject or a total dose administered to a subject irrespective to their body weight.

In one embodiment, a therapeutically effective amount of the pharmaceutical formulation according to the present invention is administrated to a patient in need thereof.

Administration is preferably in a “therapeutically effective amount”, this being sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage etc, is within the responsibility of medical doctors. Appropriate doses of antibody are well known in the art (Ledermann J A et al., (1999) Int J Cancer 47: 659-664; Bagshawe K D et al., (1991) Antibody, Immunoconjugates and Radiopharmaceuticals, 4: 915-922). The precise dose will depend upon a number of factors, including the size and location of the area to be treated, body weight of the subject, the precise nature of the antibody (e.g. whole antibody or fragment) and any additional therapeutic agents administered before, at the time of or after administration of the antibody.

The present invention also relates to a method of manufacturing the pharmaceutical formulation described herein.

The present invention also relates to a method to test the pharmaceutical formulation disclosed herein to any assay for stability determination.

The present invention also relates to an article of manufacture comprising the pharmaceutical formulation.

### EXAMPLE

### Example 1

The stability of a high concentration drug products (HC DP) lyophilizate in vial GLEc013 composed of 240 mg of Antibody 1, 4 mg L-Histidine, 200 mg Sucrose and 0.5 mg Polysorbate 80 has been tested, based on the results in Table 1 to 3 it can be concluded that Antibody 1 is stable:

1) at +5±3° C. for 48 months
2) at +25±2° C. for 12 months
3) at +40±2° C. for 3 months.

TABLE 1
Stability at Long Term Stability Condition of +5 ± 3° C.

| Stability | Specifi- | Time Interval (months) | | | | | |
|---|---|---|---|---|---|---|---|
| Tests | cation | T0 | T1 M | T2 M | T3 M | T6 M | T9 M |
| Cake | Loose | Uniform, | Uniform, | Uniform, | Uniform, | Uniform, | Uniform, |
| Appearance | powder to | flat, not | flat, | flat, | flat, not | flat, not | flat, not |
| | well- | adherent | adherent to | adherent to | adherent to | adherent to | adherent to |
| | formed | to vial | most part of | most part of | vial surface. | vial surface, | vial surface, |
| | cake, | surface, | the vial | the vial | No visible | cracked | not cracked |
| | absence of | cracked in | surface. | surface. | cracks | | |
| | foreign | some parts | Cracked in | Cracked in | | | |
| | body, no | of the | some parts | some parts | | | |
| | evidence of | surface. | of the | of the | | | |
| | meltback | | surface. | surface. | | | |
| Reconstitution | ≤30 mins | 15 min | 31 min | 6 min | 14 min | 10 min | 10 min |
| time | | | | | | | |
| Karl Fisher | ≤4% | 0.3 | NA | NA | NA | NA | NA |
| pH | pH at T0 ±0.2 | 5.9 | 6.0 | 6.0 | 6.0 | 6.0 | 6.0 |
| | ≥5.7 and ≤6.1 | | | | | | |
| Physical | Clear to | Clear and | Clear and | Clear and | Clear and | Clear and | Clear and |
| Appearance | slightly | slightly | slightly | brownish | slightly | brownish | slightly |
| of liquid | opalescent, | brownish | brownish | | brownish | | brownish |
| | colorless to | | | | | | |
| | (slightly) | | | | | | |
| | brownish/ | | | | | | |
| | yellowish | | | | | | |
| A280 | ≥120 mg/ml | 156 | 153 | 158 | 163 | 159 | 153 |
| | and ≤180 mg/ml | | | | | | |
| SE-HPLC | % monomer | 99 | 99 | 99 | 99 | 99 | 99 |
| | IgG peak ≥95% | | | | | | |
| cGE (NR) | IgG peak ≥90% | 92 | 92 | 92 | 92 | 92 | 93 |
| | Assigned | 7 | 6 | 6 | 6 | 7 | 5 |
| | peaks ≤8% | | | | | | |
| | Total other | 2 | 2 | 2 | 2 | 2 | 1 |
| | peaks ≤3% | | | | | | |
| cGE (R) | Similar | Similar | Similar | Similar | Similar | Similar | Similar |
| | profile | profile | profile | profile | profile | profile | profile |
| | to that | to that | to that | to that | to that | to that | to that |
| | of the | of the | of the | of the | of the | of the | of the |
| | Ref. Std. | Ref. Std. | Ref. Std. | Ref. Std. | Ref. Std. | Ref. Std. | Ref. Std. |
| | Heavy chain | 65 | 64 | 63 | 63 | 64 | 63 |
| | peak ≥60% | | | | | | |
| | Light chain | 35 | 35 | 36 | 35 | 34 | 35 |
| | peak ≥30% | | | | | | |
| | Total other | 1 | 1 | 1 | 2 | 3 | 3 |
| | peaks ≤3% | | | | | | |
| cIEF/iCE3 | Similar | Similar | Similar | Similar | Similar | Similar | Similar |
| | profile | profile | profile | profile | profile | profile | profile |
| | to that | to that | to that | to that | to that | to that | to that |
| | of the | of the | of the | of the | of the | of the | of the |
| | Ref. Std. | Ref. Std. | Ref. Std. | Ref. Std. | Ref. Std) | Ref. Std. | Ref. Std. |
| | Acidic | 44 | 44 | 45 | 44 | 48 | 44 |
| | peaks: ≥14% | | | | | | |
| | and ≤59% | | | | | | |
| | Main | 39 | 39 | 38 | 39 | 36 | 39 |
| | peak: ≥33% | | | | | | |
| | and ≤54% | | | | | | |
| | Basic | 17 | 17 | 17 | 17 | 16 | 17 |
| | peaks: ≥7% | | | | | | |
| | and ≤35% | | | | | | |
| | pl main | 8.3 | 8.3 | 8.3 | 8.3 | 8.3 | 8.2 |
| | peak: ≥8.1 | | | | | | |
| | and ≤8.5 | | | | | | |
| CEX-HPLC | Similar | Similar | NA | NA | NA | Similar | NA |
| | profile | profile | | | | profile | |
| | to that | to that | | | | to that | |
| | of the | of the | | | | of the | |
| | Ref. Std. | Ref. Std. | | | | Ref. Std. | |
| Binding | ≥50 and ≤150% | 82 | 142 | 122 | 123 | 127 | 97 |
| ELISA | of Ref. Std. | | | | | | |
| | EC₅₀ | | | | | | |
| ELISA Ki | Report result | 76 | NA | NA | NA | 114 | NA |
| (competition) | as % IC₅₀ of | | | | | | |
| | Ref. Std. | | | | | | |
| Subvisible | ≤6000 | 11 | NA | NA | NA | NA | NA |
| particles | particles of | | | | | | |
| (SVPs) | 10 μm in | | | | | | |
| | size or | | | | | | |
| | larger per | | | | | | |
| | container | | | | | | |
| | ≤600 | 0 | NA | NA | NA | NA | NA |
| | particles of | | | | | | |
| | 25 μm in | | | | | | |
| | size or | | | | | | |
| | larger per | | | | | | |
| | container | | | | | | |
| Container | Report | NA | NA | NA | NA | NA | NA |
| Closure | result | | | | | | |
| Integrity | | | | | | | |

| Stability | | | | | | |
|---|---|---|---|---|---|---|
| Indicating | | Time Interval (months) | | | | |
| Tests | Specification | T12 M | T18 M | T24 M | T36 M | T48 M |
| Cake | Loose powder | Uniform, | Partially | Well-formed | Well-formed | Loose powder |
| Appearance | to well- | concave, | collapsed | cake, no | cake, no | cake, no |
| | formed cake, | not adherent | cake, absence | evidence of | evidence of | evidence of |
| | absence of | to vial | of foreign | meltback, | meltback, | meltback, |
| | foreign | surface, | body, no | absence of | absence of | absence of |
| | body, no | cracked | evidence of | foreign body | foreign body | foreign body |
| | evidence of | | meltback | | | |
| | meltback | | | | | |
| Reconstitution | ≤30 mins | 5 min | 5 min | 16 min | 18 min | 14 min |
| time | | | | | | |
| Karl Fisher | ≤4% | 1 | NA | 1 | 0 | 1 |
| pH | pH at T0 ±0.2 | 6.0 | 6.0 | 6.0 | 6.0 | 6.0 |
| | ≥5.7 and ≤6.1 | | | | | |
| Physical | Clear to | Clear and | Clear and | Clear and | Slightly | Slightly |
| Appearance | slightly | brownish | brownish | brownish | opalescent, | opalescent, |
| of liquid | opalescent, | | | | brownish | brownish |
| | colorless to | | | | | |
| | (slightly) | | | | | |
| | brownish/ | | | | | |
| | yellowish | | | | | |
| A280 | ≥120 mg/ml | 148 | 157 | 150 | 165 | 146 |
| | and ≤180 mg/ml | | | | | |
| SE-HPLC | % monomer | 99 | 99 | 99 | 98 | 98 |
| | IgG peak ≥95% | | | | | |
| cGE (NR) | IgG peak ≥90% | 92 | 92 | 91 | 90 | 90 |
| | Assigned | 6 | 7 | 7 | 7 | 8 |
| | peaks ≤8% | | | | | |
| | Total other | 2 | 2 | 2 | 3 | 2 |
| | peaks ≤3% | | | | | |
| cGE (R) | Similar | Similar | Similar | Similar | Similar | Similar |
| | profile | profile | profile | profile | profile | profile |
| | to that | to that | to that | to that | to that | to that |
| | of the | of the | of the | of the | of the | of the |
| | Ref. Std. | Ref. Std. | Ref. Std. | Ref. Std. | Ref. Std. | Ref. Std. |
| | Heavy chain | 63 | 64 | 64 | 63 | 65 |
| | peak ≥60% | | | | | |
| | Light chain | 35 | 35 | 35 | 36 | 34 |
| | peak ≥30% | | | | | |
| | Total other | 2 | 1 | 1 | 1 | 1 |
| | peaks ≤3% | | | | | |
| cIEF/iCE3 | Similar | Similar | Similar | Similar | Similar | Similar |
| | profile | profile | profile | profile | profile | profile |
| | to that | to that | to that | to that | to that | to that |
| | of the | of the | of the | of the | of the | of the |
| | Ref. Std. | Ref. Std. | Ref. Std. | Ref. Std. | Ref. Std. | Ref. Std. |
| | Acidic | 42 | 44 | 45 | 46 | 44 |
| | peaks: ≥14% | | | | | |
| | and ≤59% | | | | | |
| | Main | 40 | 39 | 38 | 37 | 38 |
| | peak: ≥33% | | | | | |
| | and ≤54% | | | | | |
| | Basic | 18 | 18 | 17 | 18 | 18 |
| | peaks: ≥7% | | | | | |
| | and ≤35% | | | | | |
| | pl main | 8.3 | 8.3 | 8.3 | 8.4 | 8.3 |
| | peak: ≥8.1 | | | | | |
| | and ≤8.5 | | | | | |
| CEX-HPLC | Similar | Similar | Similar | Similar | Similar | Similar |
| | profile | profile | profile | profile | profile | profile |
| | to that | to that | to that | to that | to that | to that |
| | of the | of the | of the | of the | of the | of the |
| | Ref. Std. | Ref. Std. | Ref. Std. | Ref. Std. | Ref. Std. | Ref. Std. |
| Binding ELISA | ≥50 and ≤150% | 111 | 99 | 97 | 83 | 113 |
| | of Ref. Std. | | | | | |
| | EC₅₀ | | | | | |
| ELISA Ki | Report result | 130 | 91 | 82 | 84 | 99 |
| (competition) | as % IC₅₀ | | | | | |
| | of Ref. Std. | | | | | |
| Subvisible | ≤6000 | 403 | NA | 98 | 43 | 81 |
| particles | particles of | | | | | |
| (SVPs) | 10 μm in | | | | | |
| | size or | | | | | |
| | larger per | | | | | |
| | container | | | | | |
| | ≤600 | 46 | NA | 26 | 22 | 48 |
| | particles of | | | | | |
| | 25 μm in | | | | | |
| | size or | | | | | |
| | larger per | | | | | |
| | container | | | | | |
| Container | Report | Pass | NA | Pass | NA | NA |
| Closure | result | | | | | |
| Integrity | | | | | | |

TABLE 2
Stability at Accelerated Stability Condition of +25 ± 2° C.

| Stability | | | | | | | | |
|---|---|---|---|---|---|---|---|---|
| Indicating | | Time Interval (months) | | | | | | |
| Tests | Specification | T0 | T1 M | T2 M | T3 M | T6 M | T9 M | T12 M |
| Cake | Loose powder | Uniform, | Uniform, | Uniform, | Uniform, | Uniform, | Uniform, | Uniform, |
| Appearance | to well- | flat, not | flat, adherent | flat, adherent | flat, not | flat, not | flat, not | concave, |
| | formed cake, | adherent | to most part | to most part | adherent to | adherent to | adherent to | not adherent |
| | absence of | to vial | of the vial | of the vial | vial surface. | vial surface, | vial surface, | to vial |
| | foreign body, | surface, | surface. | surface. | No visible | cracked | cracked in | surface, |
| | no evidence | cracked in | Cracked in | Cracked in | cracks. | | some parts of | cracked |
| | of meltback | some parts | some parts | some parts | | | the surface. | |
| | | of the | of the | of the | | | | |
| | | surface. | | | | | | |
| Reconstitution | ≤30 mins | 15 min | 21 mi | 5 min | 13 min | 26 min | 8 min | 7 min |
| time | | | | | | | | |
| Karl Fisher | ≤4% | 0.3 | NA | NA | NA | NA | NA | 1 |
| pH | pH at T0 ±0.2 | 5.9 | 6.0 | 6.0 | 6.0 | 6.0 | 6.0 | 6.0 |
| | (≥5.7 and ≤6.1) | | | | | | | |
| Appearance | Clear to | Clear and | Clear and | Clear and | Clear and | Slightly | Clear and | Clear and |
| (liquid) | slightly | slightly | slightly | brownish | slightly | opalescent | slightly | brownish |
| | opalescent, | brownish | brownish | | brownish | and | brownish | |
| | colorless to | | | | | brownish | | |
| | (slightly) | | | | | | | |
| | brownish/ | | | | | | | |
| | yellowish | | | | | | | |
| A280 | ≥120 mg/ml | 156 | 155 | 168 | 163 | 162 | 149 | 143 |
| | and ≤180 mg/ml | | | | | | | |
| SE-HPLC | % monomer | 99 | 99 | 98 | 99 | 99 | 98 | 98 |
| | IgG peak ≥95% | | | | | | | |
| cGE (NR) | IgG peak ≥90% | 92 | 92 | 92 | 92 | 92 | 93 | 92 |
| | Assigned | 7 | 6 | 7 | 7 | 6 | 6 | 6 |
| | peaks ≤8% | | | | | | | |
| | Total other | 2 | 2 | 2 | 2 | 2 | 2 | 2 |
| | peaks ≤3% | | | | | | | |
| cGE (R) | Similar | Similar | Similar | Similar | Similar | Similar | Similar | Similar |
| | profile | profile | profile | profile | profile | profile | profile | profile |
| | to that | to that | to that | to that | to that | to that | to that | to that |
| | of the | of the | of the | of the | of the | of the | of the | of the |
| | Ref. Std. | Ref. Std. | Ref. Std. | Ref. Std. | Ref. Std. | Ref. Std. | Ref. Std. | Ref. Std. |
| | Heavy chain | 65 | 65 | 63 | 63 | 64 | 63 | 64 |
| | peak ≥60% | | | | | | | |
| | Light chain | 35 | 35 | 36 | 36 | 34 | 35 | 34 |
| | peak ≥30% | | | | | | | |
| | Total other | 1 | 1 | 1 | 2 | 2 | 3 | 2 |
| | peaks ≤3% | | | | | | | |
| cIEF/iCE3 | Similar | Similar | Similar | Similar | Similar | Similar | Similar | Similar |
| | profile | profile | profile | profile | profile | profile | profile | profile |
| | to that | to that | to that | to that | to that | to that | to that | to that |
| | of the | of the | of the | of the | of the | of the | of the | of the |
| | Ref. Std. | Ref. Std. | Ref. Std. | Ref. Std. | Ref. Std. | Ref. Std. | Ref. Std. | Ref. Std. |
| | Acidic | 44 | 43 | 45 | 44 | 48 | 45 | 43 |
| | peaks: ≥14% | | | | | | | |
| | and ≤59% | | | | | | | |
| | Main | 39 | 40 | 38 | 38 | 36 | 36 | 38 |
| | peak: ≥33% | | | | | | | |
| | and ≤54% | | | | | | | |
| | Basic | 17 | 18 | 17 | 18 | 16 | 19 | 19 |
| | peaks: ≥7% | | | | | | | |
| | and ≤35% | | | | | | | |
| | pl main | 8.3 | 8.4 | 8.3 | 8.3 | 8.3 | 8.3 | 8.3 |
| | peak: ≥8.1 | | | | | | | |
| | and ≤8.5 | | | | | | | |
| CEX-HPLC | Similar | Similar | NA | NA | NA | Similar | NA | Similar |
| | profile | profile | | | | profile | | profile |
| | to that | to that | | | | to that | | to that |
| | of the | of the | | | | of the | | of the |
| | Ref. Std. | Ref. Std. | | | | Ref. Std. | | Ref. Std. |
| Binding | ≥50 and ≤150% | 82 | 112 | 121 | 119 | 88 | 69 | 111 |
| ELISA | of Ref. Std. | | | | | | | |
| | EC₅₀ | | | | | | | |
| ELISA Ki | Report result | 76 | NA | NA | NA | 110 | NA | 102 |
| (competition) | as % IC₅₀ | | | | | | | |
| | of Ref. Std. | | | | | | | |
| Particulate | ≤6000 | 11 | NA | NA | NA | NA | NA | 13 |
| content | particles of | | | | | | | |
| (Subvisible) | 10 μm in | | | | | | | |
| | size or | | | | | | | |
| | larger per | | | | | | | |
| | container | | | | | | | |
| | ≤600 | 0 | NA | NA | NA | NA | NA | 0 |
| | particles of | | | | | | | |
| | 25 μm in | | | | | | | |
| | size or | | | | | | | |
| | larger per | | | | | | | |
| | container | | | | | | | |

TABLE 3
Stability at Stress Stability Condition of +40 ± 2° C.

| Stability | | | | | |
|---|---|---|---|---|---|
| Indicating | | Time Interval (months) | | | |
| Tests | Specification | T0 | T1 M | T2 M | T3 M |
| Cake | Loose powder to well- | Uniform, | Uniform, flat, | Uniform, flat, | Uniform, flat, |
| Appearance | formed cake, absence | flat, not | adherent to | adherent to | not adherent |
| | of foreign body, no | adherent | most part of | most part of | to vial |
| | evidence of meltback | to vial | the vial | the vial | surface. |
| | | surface, | surface. | surface. | Cracked |
| | | cracked in | Cracked in | Cracked in | |
| | | some parts | some parts | some parts | |
| | | of the | of the | of the | |
| | | surface. | surface. | surface. | |
| Reconstitution | ≤30 mins | 15 min | 23 min | 6 min | 13 min |
| time | | | | | |
| Karl Fisher | ≤4% | 0.3% | NA | NA | 1% |
| pH | pH at T0 ± 0.2 | 5.9 | 6.0 | 6.0 | 6.0 |
| | (≥5.7 and ≤6.1) | | | | |
| Appearance | Clear to slightly | Clear and | Clear and | Clear and | Clear and |
| (liquid) | opalescent, colorless | slightly | slightly | brownish | slightly |
| | to (slightly) | brownish | brownish | | brownish |
| | brownish/yellowish | | | | |
| A₂₈₀ | ≥120 mg/ml and ≤180 | 156 | 155 | 162 | 163 |
| | mg/ml | | | | |
| SE-HPLC | % monomer IgG peak ≥ | 99 | 98 | 97 | 97 |
| | 95% | | | | |
| cGE (NR) | IgG peak ≥ 90% | 92 | 92 | 92 | 91 |
| | Assigned peaks ≤ 8% | 7 | 6 | 6 | 7 |
| | Total other peaks ≤ 3% | 2 | 2 | 2 | 2 |
| cGE (R) | Similar profile to that | Similar | Similar | Similar | Similar |
| | of the Ref. Std. | profile to | profile to | profile to | profile to |
| | | that of the | that of the | that of the | that of the |
| | | Ref. Std. | Ref. Std. | Ref. Std. | Ref. Std. |
| | Heavy chain peak ≥ | 65 | 64 | 63 | 62 |
| | 60% | | | | |
| | Light chain peak ≥ 30% | 35 | 35 | 36 | 36 |
| | Total other peaks ≤ 3% | 1 | 1 | 1 | 2 |
| cIEF/iCE3 | Similar profile to that | Similar | Similar | Similar | Similar |
| | of the Ref. Std. | profile to | profile to | profile to | profile to |
| | | that of the | that of the | that of the | that of the |
| | | Ref. Std. | Ref. Std. | Ref. Std. | Ref. Std. |
| | Acidic peaks: ≥14% | 44 | 43 | 45 | 44 |
| | and ≤59% | | | | |
| | Main peak: ≥33% | 39 | 38 | 36 | 37 |
| | and ≤54% | | | | |
| | Basic peaks: ≥7% and | 17 | 19 | 18 | 19 |
| | ≤35% | | | | |
| | pl main peak: ≥8.1 | 8.3 | 8.3 | 8.3 | 8.3 |
| | and ≤8.5 | | | | |
| CEX-HPLC | Similar profile to that | Similar | NA | NA | Similar |
| | of the Ref. Std. | profile to | | | profile to |
| | | that of the | | | that of the |
| | | Ref. Std. | | | Ref. Std. |
| Binding ELISA | ≥50 and ≤150% of | 82% | 120 | 106 | 109 |
| | Ref. Std. EC₅₀ | | | | |
| ELISA Ki | Report result as % IC₅₀ | 76 | NA | NA | 95% |
| (competition) | of Ref. Std. | | | | |
| Particulate | ≤6000 particles of | 11 | NA | NA | 0 |
| content | 10 μm in size or larger | | | | |
| (Subvisible) | per container | | | | |
| | ≤600 particles of | 0 | NA | NA | 0 |
| | 25 μm in size or larger | | | | |
| | per container | | | | |

### Example 2

Next the stability of a second presentation of the high concentration drug products (HC DP) lyophilizate in vial GLEc013 composed of 192 mg of Antibody 1, 3.1 mg L-Histidine, 160 mg Sucrose and 0.4 mg Polysorbate 80 was investigated. Based on the results in Table 4 to 6 it can be concluded that Antibody 1 is stable for:
1) 24 months when stored at +5±3° C.
2) 12 months when stored at +25±2° C./60%±5% RH.
3) 3 months when stored at +40±2° C./75%±5% RH.

TABLE 4
Stability at Stability Condition of +5 ± 3° C.

| | | Time interval (months) | | | | | | |
|---|---|---|---|---|---|---|---|---|
| Stability | | Initial | | | | | | |
| Tests | Specification | T0 | T3 | T6 | T9 | T12 | T18 | T24 |
| Physical | Clear to | Clear and | Clear and | Clear and | Clear and | Clear and | Clear and | Clear and |
| appearance | slightly | slightly | slightly | slightly | slightly | slightly | slightly | slightly |
| of liquid | opalescent, | yellowish | yellowish | yellowish | yellowish | yellowish | brownish. | brownish. |
| | colorless to | liquid. | liquid. | liquid. | liquid. | liquid. | Practically | Practically |
| | brownish/ | Practically | Practically | Practically | Practically | Practically | free of | free of |
| | yellowish, | free of | free of | free of | free of | free of | visual | visual |
| | practically | visual | visual | visual | visual | visual | particulates | particulates |
| | free of | particulates. | particulates. | particulates. | particulates. | particulates. | | |
| | particulates | | | | | | | |
| pH | pH at T0 ±0.2 | 6.2 | 6.3 | 6.2 | 6.2 | 6.2 | 6.2 | 6.2 |
| | (≥6.0 and ≤6.4) | | | | | | | |
| Cake | Loose powder | Uniform, | Well-formed | Well-formed | Well-formed | Well-formed | Well-formed | Well-formed |
| appearance | to well- | flat, | cake, | cake, | cake, | cake, | cake, | cake, |
| | formed cake, | adherent | absence | absence | absence | absence | absence | absence |
| | absence of | to the vial | of foreign | of foreign | of foreign | of foreign | of foreign | of foreign |
| | foreign body, | surface, | body, no | body, no | body, no | body, no | body, no | body, no |
| | no evidence | some cracks | evidence of | evidence of | evidence of | evidence of | evidence of | evidence of |
| | of meltback | | meltback | meltback | melt back | melt back | melt back | melt back |
| | (2) | | | | | | | |
| Reconstitution | ≤30 mins | 4 min 10 sec | 6 min 30 sec | 15 min 25 sec | 12 min 36 sec | 7 min 32 sec | 17 min 27 sec | 11 min 40 sec |
| time | | | | | | | | |
| Karl Fisher | ≤4% | 1 | NA | NA | NA | 1 | NA | 1 |
| A₂₈₀ | Concentration | 151 | 145 | 145 | 144 | 141 | 148 | 145 |
| | at T0 ±10% | | | | | | | |
| | (≥136 mg/mL and | | | | | | | |
| | ≤166 mg/mL) | | | | | | | |
| SE-HPLC | % monomer | 99 | 99 | 98 | 99 | 98 | 98 | 99 |
| | IgG peak ≥95% | | | | | | | |
| CGE (non- | IgG peak ≥90% | 93 | 92 | 93 | 92 | 93 | 93 | 93 |
| reduced) | | | | | | | | |
| | Assigned | 5 | 6 | 5 | 6 | 6 | 4 | 5 |
| | peaks ≤8% | | | | | | | |
| | Total other | 2 | 1 | 2 | 2 | 2 | 2 | 2 |
| | peaks ≤3% | | | | | | | |
| CGE | Similar | Similar | Similar | Similar | Similar | Similar | Similar | Similar |
| (reduced) | profile | profile | profile | profile | profile | profile | profile | profile |
| | to that | to that | to that | to that | to that | to that | to that | to that |
| | of the | of the | of the | of the | of the | of the | of the | of the |
| | Ref. Std. | Ref. Std. | Ref. Std. | Ref. Std. | Ref. Std. | Ref. Std. | Ref. Std. | Ref. Std. |
| | Heavy chain | 64 | 64 | 65 | 64 | 64 | 64 | 63 |
| | peak ≥60% | | | | | | | |
| | Light chain | 35 | 35 | 35 | 35 | 35 | 35 | 36 |
| | peak ≥30% | | | | | | | |
| | Total other | 1 | 1 | 1 | 1 | 1 | 1 | 1 |
| | peaks ≤3% | | | | | | | |
| cIEF/iCE3 | Similar | Similar | Similar | Similar | Similar | Similar | Similar | Similar |
| | profile | profile | profile | profile | profile | profile | profile | profile |
| | to that | to that | to that | to that | to that | to that | to that | to that |
| | of the | of the | of the | of the | of the | of the | of the | of the |
| | Ref. Std. | Ref. Std. | Ref. Std. | Ref. Std. | Ref. Std. | Ref. Std. | Ref. Std. | Ref. Std. |
| | Acidic | 40 | 33 | 33 | 35 | 34 | 33 | 33 |
| | peaks: ≥14% | | | | | | | |
| | and ≤59% | | | | | | | |
| | Main | 41 | 45 | 45 | 44 | 44 | 45 | 45 |
| | peak: ≥33% | | | | | | | |
| | and ≤54% | | | | | | | |
| | Basic | 20 | 22 | 22 | 21 | 22 | 22 | 23 |
| | peaks: ≥7% | | | | | | | |
| | and ≤35% | | | | | | | |
| | pl main | 8.3 | 8.3 | 8.3 | 8.3 | 8.3 | 8.3 | 8.2 |
| | peak: ≥8.1 | | | | | | | |
| | and ≤8.5 | | | | | | | |
| HPLC-CEX | Similar | Similar | NA | Similar | NA | Similar | Similar | Similar |
| | profile | profile | | profile | | profile | profile | profile |
| | to that | to that | | to that | | to that | to that | to that |
| | of the | of the | | of the | | of the | of the | of the |
| | Ref. Std. | Ref. Std. | | Ref. Std. | | Ref. Std. | Ref. Std. | Ref. Std. |
| | Report | 18 | NA | 21 | NA | 20 | 19 | 18 |
| | % acidic | | | | | | | |
| | Report | 47 | NA | 49 | NA | 50 | 49 | 48 |
| | % main | | | | | | | |
| | Report | 35 | NA | 30 | NA | 30 | 32 | 31 |
| | % basic | | | | | | | |
| Binding | ≥50% and ≤150% | 123 | 75 | 80 | 104 | 102 | 80 | 105 |
| ELISA | of Ref. Std. | | | | | | | |
| | EC₅₀ | | | | | | | |
| ELISA Ki | Report result | 89 | NA | 100 | NA | 94 | 117 | 76 |
| (competition) | as % IC₅₀ | | | | | | | |
| | of Ref. Std. | | | | | | | |
| Subvisible | Report 2 μm | NA | NA | NA | NA | 600 | NA | 319 |
| particles | in size or | | | | | | | |
| | larger per | | | | | | | |
| | container | | | | | | | |
| | Report 5 μm | NA | NA | NA | NA | 55 | NA | 119 |
| | in size or | | | | | | | |
| | larger per | | | | | | | |
| | container | | | | | | | |
| | ≤6000 | 27 | NA | NA | NA | 11 | NA | 84 |
| | particles of | | | | | | | |
| | 10 μm in size | | | | | | | |
| | or larger per | | | | | | | |
| | container | | | | | | | |
| | ≤600 | 1 | NA | NA | NA | 0 | NA | 33 |
| | particles of | | | | | | | |
| | 25 μm in size | | | | | | | |
| | or larger per | | | | | | | |
| | container | | | | | | | |
| Container | Report | NA | NA | NA | NA | Pass | NA | Pass |
| closure | results | | | | | | | |
| integrity | | | | | | | | |
| Sterility | No Growth | No growth | NA | NA | NA | NA | NA | NA |
| | at 14 days | at 14 days | | | | | | |

TABLE 5
Stability at Stability Condition of +25 ± 2° C./60% ± 5% RH

| Stability | | Time Interval (months) | | | | |
|---|---|---|---|---|---|---|
| Tests | Specification | Initial T0 | T3 | T6 | T9 | T12 |
| Physical | Clear to | Clear and | Clear and | Clear and | Clear and | Clear and |
| appearance | slightly | slightly | slightly | slightly | slightly | slightly |
| of liquid | opalescent, | yellowish | yellowish | yellowish | yellowish | yellowish |
| | colorless to | liquid. | liquid. | liquid. | liquid. | liquid. |
| | brownish/ | Practically | Practically | Practically | Practically | Practically |
| | yellowish, | free of | free of | free of | free of | free of |
| | practically | visual | visual | visual | visual | visual |
| | free of | particulates. | particulates. | particulates. | particulates. | particulates. |
| | particulates | | | | | |
| pH | pH at T0 ± 0.2 | 6.2 | 6.2 | 6.2 | 6.2 | 6.2 |
| | (≥6.0 and ≤6.4) | | | | | |
| Cake | Loose powder | Uniform, flat, | Uniform cake, | Uniform cake, | Well-formed | Well-formed |
| appearance | to well- | adherent to | absence of | absence of | cake, absence | cake, absence |
| | formed cake, | the vial | foreign | foreign | of foreign | of foreign |
| | absence of | surface, | body, no | body, no | body, no | body, no |
| | foreign body, | some cracks | evidence of | evidence | evidence | evidence |
| | no evidence | | meltback, | of melt- | of meltback | of meltback |
| | of meltback (2) | | cracked. | back | | |
| Reconstitution | ≤30 mins | 4 min 10 sec | 7 min 22 sec | 16 min 11 sec | 13 min 22sec | 8 min 46 sec |
| time | | | | | | |
| Karl Fisher | ≤4% | 1 | NA | NA | NA | 1 |
| A₂₈₀ | Concentration at | 151 | 144 | 147 | 142 | 144 |
| | T0 ±10% | | | | | |
| | (≥136 mg/mL | | | | | |
| | and ≤166 mg/mL) | | | | | |
| SE-HPLC | % monomer IgG | 99 | 96 | 98 | 98 | 97 |
| | peak ≥95% | | | | | |
| CGE (non- | IgG peak ≥90% | 93 | 93 | 94 | 92 | 93 |
| reduced) | | | | | | |
| | Assigned | 5 | 5 | 5 | 6 | 6 |
| | peaks ≤8% | | | | | |
| | Total other | 2 | 1 | 2 | 2 | 2 |
| | peaks ≤3% | | | | | |
| CGE | Similar | Similar | Similar | Similar | Similar | Similar |
| (reduced) | profile | profile | profile | profile | profile | profile |
| | to that | to that | to that | to that | to that | to that |
| | of the | of the | of the | of the | of the | of the |
| | Ref. Std. | Ref. Std. | Ref. Std. | Ref. Std. | Ref. Std. | Ref. Std. |
| | Heavy chain | 64 | 64 | 64 | 64 | 63 |
| | peak ≥60% | | | | | |
| | Light chain | 35 | 35 | 35 | 35 | 35 |
| | peak ≥30% | | | | | |
| | Total other | 1 | 1 | 1 | 1 | 1 |
| | peaks ≤3% | | | | | |
| cIEF/iCE3 | Similar | Similar | Similar | Similar | Similar | Similar |
| | profile | profile | profile | profile | profile | profile |
| | to that | to that | to that | to that | to that | to that |
| | of the | of the | of the | of the | of the | of the |
| | Ref. Std. | Ref. Std. | Ref. Std. | Ref. Std. | Ref. Std. | Ref. Std. |
| | Acidic | 40 | 33 | 34 | 34 | 34 |
| | peaks: ≥14% | | | | | |
| | and ≤59% | | | | | |
| | Main | 41 | 42 | 43 | 43 | 42 |
| | peak: ≥33% | | | | | |
| | and ≤54% | | | | | |
| | Basic | 20 | 25 | 23 | 23 | 24 |
| | peaks: ≥7% | | | | | |
| | and ≤35% | | | | | |
| | pl main | 8.3 | 8.3 | 8.3 | 8.3 | 8.3 |
| | peak: ≥8.1 | | | | | |
| | and ≤8.5 | | | | | |
| HPLC-CEX | Similar | Similar | NA | Similar | NA | Similar |
| | profile | profile | | profile | | profile |
| | to that | to that | | to that | | to that |
| | of the | of the | | of the | | of the |
| | Ref. Std. | Ref. Std. | | Ref. Std. | | Ref. Std. |
| | Report % | 18 | NA | 21 | NA | 20 |
| | acidic | | | | | |
| | Report % | 47 | NA | 49 | NA | 49 |
| | main | | | | | |
| | Report % | 35 | NA | 30 | NA | 31 |
| | basic | | | | | |
| Binding | ≥50% and ≤150 | 123 | 73 | 89 | 102 | 87 |
| ELISA | % of Ref. Std. | | | | | |
| | EC₅₀ | | | | | |
| ELISA Ki | Report result | 89 | NA | 112 | NA | 123 |
| (competition) | as % IC₅₀ | | | | | |
| | of Ref. Std. | | | | | |
| Sub-visible | Report 2 μm | NA | NA | NA | NA | 825 |
| particles | in size or | | | | | |
| | larger per | | | | | |
| | container | | | | | |
| | Report 5 μm | NA | NA | NA | NA | 70 |
| | in size or | | | | | |
| | larger per | | | | | |
| | container | | | | | |
| | ≤6000 | 27 | NA | NA | NA | 28 |
| | particles | | | | | |
| | of 10 μm | | | | | |
| | in size or | | | | | |
| | larger per | | | | | |
| | container | | | | | |
| | ≤600 | 1 | NA | NA | NA | 0 |
| | particles | | | | | |
| | of 25 μm | | | | | |
| | in size or | | | | | |
| | larger per | | | | | |
| | container | | | | | |

TABLE 6
Stability at Stability Condition of +40 ± 2° C./75% ± 5% RH

| Stability | | | |
|---|---|---|---|
| Indicating | | Time Interval (months) | |
| Tests | Specification | Initial T0 | T3 |
| Physical | Clear to slightly opalescent, | Clear and slightly | Clear and slightly |
| appearance of | colorless to | yellowish liquid. | yellowish liquid. |
| liquid | brownish/yellowish, | Practically free of visual | Practically free of visual |
| | practically free of | particulates. | particulates. |
| | particulates | | |
| pH | pH at T0 ± 0.2 (≥6.0 and ≤ | 6.2 | 6.2 |
| | 6.4) | | |
| Cake | Loose powder to well-formed | Uniform, flat, adherent | Uniform cake, absence |
| appearance | cake, absence of foreign | to the vial surface, some | of foreign body, no |
| | body, no evidence of melt- | cracks | evidence of melt-back. |
| | back (2) | | |
| Reconstitution | ≤30 mins | 4 min 10 sec | 7 min 04 sec |
| time | | | |
| Karl Fisher | ≤4% | 1 | 1 |
| A280 | Concentration at T0 ± 10% (≥ | 151 | 150 |
| | 136 mg/mL and ≤166 | | |
| | mg/mL) | | |
| SE-HPLC | % monomer IgG peak ≥ 95% | 99 | 98 |
| CGE (non- | IgG peak ≥ 90% | 93 | 93 |
| reduced) | Assigned peaks ≤ 8% | 5 | 6 |
| | Total other peaks ≤ 3% | 2 | 1 |
| CGE (reduced) | Similar profile to that of the | Similar profile to that of | Similar profile to that of |
| | Ref. Std. | the Ref. Std. | the Ref. Std. |
| | Heavy chain peak ≥ 60% | 64 | 63 |
| | Light chain peak ≥ 30% | 35 | 34 |
| | Total other peaks ≤ 3% | 1 | 1 |
| cIEF/iCE3 | Similar profile to that of the | Similar profile to that of | Similar profile to that of |
| | Ref. Std. | the Ref. Std. | the Ref. Std. |
| | Acidic peaks: ≥ 14% and ≤ | 40 | 33 |
| | 59% | | |
| | Main peak: ≥33% and ≤54% | 41 | 44 |
| | Basic peaks: ≥7% and ≤35% | 20 | 23 |
| | pl main peak: ≥8.1 and ≤8.5 | 8.3 | 8.3 |
| HPLC-CEX | Similar profile to that of the | Similar profile to that of | Similar profile to that of |
| | Ref. Std. | the Ref. Std. | the Ref. Std. |
| | Report % acidic | 18 | 30 |
| | Report % main | 47 | 47 |
| | Report % basic | 35 | 23 |
| Binding ELISA | ≥50% and ≤150% of Ref. Std. | 123 | 93 |
| | EC₅₀ | | |
| ELISA Ki | Report result as % IC₅₀ of Ref. | 89 | 88 |
| (Competition) | Std. | | |
| Sub-visible | Report 2 μm in size or larger | NA | 149 |
| particles | per container | | |
| | Report 5 μm in size or larger | NA | 0 |
| | per container | | |
| | ≤6000 particles of 10 μm in | 27 | 0 |
| | size or larger per container | | |
| | ≤600 particles of 25 μm in | 1 | 0 |
| | size or larger per container | | |

### Example 3

The comparability of Antibody 1 with syringes and infusion bags, used in the clinics for subcutaneous (SC) injection and for intravenous (IV) infusion, respectively, was also investigated with regards to protein stability and adsorption over time at different temperatures and relevant protein concentrations.

### SC Injection

The lyophilized drug product with each vials is composed of 240 mg Antibody 1, 4 mg Histidine, 200 mg sucrose and 0.5 mg polysorbate 80 (as in Example 1), the stability of Antibody 1 was reconstituted with water for injection (WFI) to target an antibody concentration of about 150 mg/ml.

Stability studies were performed in fully filled 2 ml syringes, in fully filled 1 mL syringes and in half filled 1 mL syringes, stored at 5±3° C. for 48 hours or at 25±2° C. for 24 hours. The results of the stability studies are shown in Table 7. The reconstituted drug product was found to be stable for 24 h at 25±2° C. and 48 h at 5±3° C. when stored in a syringe.

TABLE 7
Stability Antibody 1 formulation for SC administration at 5 ± 3° C. and 25 ± 2° C.

| | Concentration and Incubation time | | |
|---|---|---|---|
| | Target antibody concentration: 150 mg/ml | | |
| | | 2 ml fill in | 1 ml fill in |
| Stability | | 2 ml syringe | 1 ml syringe |

| Indicating | Specifica- | | 48 h at | 24 h at | 48 h at |
|---|---|---|---|---|---|
| Tests | tion | T0 | 5 ± 3° C. | 25 ± 2° C. | 5 ± 3° C. |
| Physical | Clear to | Clear and | Clear and | Clear and | Clear and |
| appearance | slightly | colorless | colorless | colorless | colorless |
| of liquid | opalescent | | | | |
| | liquid, | | | | |
| | colorless to | | | | |
| | slightly | | | | |
| | yellowish | | | | |
| | liquid | | | | |
| pH | Report | 5.972 | 5.967 | 5.965 | 5.963 |
| | results | | | | |
| A280 | T0 +/−20% | 11.125 | 8.592 | 10.553 | 8.457 |
| | (mg/ml) | | | | |

| | | | |
|---|---|---|---|
| 9.367 | 9.700 | 9.171 | 9.417 |

| | | | | | |
|---|---|---|---|---|---|
| SE-HPLC | % monomer ≥98% | 99% | 99% | 99% | 99% |
| iCE | Comparable | Conform | Conform | Conform | Conform |
| | to reference | | | | |
| | standard | | | | |
| Binding | 100 ± 50% | 85% | 85% | 89% | 111% |
| ELISA | of the | | | | |
| | reference | | | | |
| | standard | | | | |
| cGE (R) | Comparable | Conform | Conform | Conform | Conform |
| | to reference | | | | |
| | standard | | | | |
| cGE (NR) | Comparable | Conform | Conform | Conform | Conform |
| | to reference | | | | |
| | standard | | | | |
| Sub-visible | Not more | Number of | Number of | Number of | Number of |
| particles | than 6000 | particles/ | particles/ | particles/ | particles/ |
| | particles/ | container ≥10 | container ≥10 | container ≥10 | container ≥10 |
| | container | μm: 80.00; | μm: 141.42; | μm: 75.72; | μm: 47.86; |
| | of size | Number of | Number of | Number of | Number of |
| | equal to or | particles/ | particles/ | particles/ | particles/ |
| | greater | container ≥25 | container ≥25 | container ≥25 | container ≥25 |
| | than 10 μm | μm: 0.00 | μm: 0.00 | μm: 0.00 | μm: 0.00 |
| | and not | | | | |
| | more than | | | | |
| | 600 | | | | |
| | particles/ | | | | |
| | container | | | | |
| | of size | | | | |
| | equal to or | | | | |
| | greater | | | | |
| | than 25 μm* | | | | |

| | | Concentration and Incubation time | | |
|---|---|---|---|---|
| | | Target antibody concentration: 150 mg/ml | | |
| | | 1 ml fill in | 0.5 ml fill in | |
| Stability | | 1 ml syringe | 1 ml syringe | |
| Indicating | Specifica- | 24 h at | 48 h at | 24 h at |
| Tests | tion | 25 ± 2° C. | 5 ± 3° C. | 25 ± 2° C. |
| Physical | Clear to | Clear and | Clear and | Clear and |
| appearance | slightly | colorless | colorless | colorless |
| of liquid | opalescent | | | |
| | liquid, | | | |
| | colorless to | | | |
| | slightly | | | |
| | yellowish | | | |
| | liquid | | | |
| pH | Report | 5.976 | 6.004 | 6.008 |
| | results | | | |
| A280 | T0 +/− 20% | 8.753 | 8.961 | 8.672 |
| | (mg/ml) | | | |
| SE-HPLC | % monomer ≥98% | 99% | 99% | 99% |
| iCE | Comparable | Conform | Conform | Conform |
| | to reference | | | |
| | standard | | | |
| Binding | 100 ± 50% | 81% | 71% | 95% |
| ELISA | of the | | | |
| | reference | | | |
| | standard | | | |
| cGE (R) | Comparable | Conform | Conform | Conform |
| | to reference | | | |
| | standard | | | |
| cGE (NR) | Comparable | Conform | Conform | Conform |
| | to reference | | | |
| | standard | | | |
| Sub-visible | Not more | Number of | Number of | Number of |
| particles | than 6000 | particles/ | particles/ | particles/ |
| | particles/ | container ≥10 | container ≥10 | container ≥10 |
| | container | μm: 32.85; | μm: 18.22; | μm: 32.86; |
| | of size | Number of | Number of | Number of |
| | equal to or | particles/ | particles/ | particles/ |
| | greater | container ≥25 | container ≥25 | container ≥25 |
| | than 10 μm | μm: 0.00 | μm: 0.36 | μm: 0.36 |
| | and not | | | |
| | more than | | | |
| | 600 | | | |
| | particles/ | | | |
| | container | | | |
| | of size | | | |
| | equal to or | | | |
| | greater | | | |
| | than 25 μm* | | | |

IV infusion

The lyophilized drug product with each vials is composed of 240 mg Antibody 1, 4 mg Histidine, 200 mg sucrose and 0.5 mg polysorbate 80 (as in Example 1), the stability of Antibody 1 was reconstituted with water for injection (WFI) to target an antibody concentration of about 150 mg/ml, and further dilution in 500 ml 0.9% NaCl infusion bags for IV infusion, to target an antibody concentration of 2.2 mg/ml.

The antibody stability was studied at 5±3° C. for 48 hours and at 25±2° C. for 24 hours. The results of the stability studies are shown in Table 8. Antibody 1 was found to be stable for 24 h at 25±2° C. and 48 h at 5±3° C. when diluted in saline bags.

TABLE 8
Stability Antibody 1 formulation for IV administration at 5 ± 3° C. and 25 ± 2° C.

| | | Concentration and Incubation time | | | |
|---|---|---|---|---|---|
| | | Target concentration: 2.2 mg/ml | | | |
| Stability | | T0 | T0 | | |
| Indicating | | (corresponding | (corresponding | 48 h at | 24 h at |
| Tests | Specification | to 5 ± 3° C.) | to 25 ± 2° C.) | 5 ± 3° C. | 25 ± 2° C. |
| Physical | Clear to slightly | Clear and | Clear and | Clear and | Clear and |
| appearance | opalescent liquid, | colorless | colorless | colorless | colorless |
| of liquid | colorless to slightly | | | | |
| | yellowish liquid | | | | |
| pH | Report results* | 6.226 | 6.336 | 6.086 | 6.110 |
| A280 | T0 +/− 20% (mg/ml) | 1.884 | 1.882 | 1.863 | 1.857 |
| SE-HPLC | % monomer ≥ 98% | 99% | 99% | 99% | 99% |
| iCE | Comparable to | Conform | Conform | Conform | Conform |
| | reference standard | | | | |
| Binding | 100 ± 50% of the | 98% | NA | 106% | 112% |
| ELISA | reference standard | | | | |
| cGE (R) | Comparable to | Conform | Conform | Conform | Conform |
| | reference standard | | | | |
| cGE (NR) | Comparable to | Conform | Conform | Conform | Conform |
| | reference standard | | | | |
| Sub-visible | ≤25 particles/mL of | Particles/mL | Particles/mL | Particles/mL | Particles/mL |
| particles | size equal to or | for ≥ 25 μm: | for ≥ 25 μm: | for ≥ 25 μm: | for ≥ 25 μm: |
| | greater than ≥ 10 μm | 48.67 | 44.83 | 10 | 5.34 |
| | & ≤ 3 particles/mL of | Particles/mL | Particles/mL | Particles/mL | Particles/mL |
| | size equal to or | for ≥ 10 μm: | for ≥ 10 μm: | for ≥ 10 μm: | for ≥ 10 μm: |
| | greater than ≥ 25 μm | 0.67 | 0.50 | 0 | 0.33 |

1. A stable pharmaceutical formulation comprising an anti-OX40 antagonist antibody or antibody fragment thereof, one or more stabilizing or tonicity agent and a buffer, wherein said pharmaceutical formulation is liquid or lyophilized.

2. The pharmaceutical formulation of any one of the preceding claims, wherein said buffer is selected from the group comprising acetate, L-histidine, citrate and phosphate and wherein said buffer is present within said pharmaceutical formulation at concentration between about 1 mM and about 10 mM.

3. The pharmaceutical formulation of any one of the preceding claims, wherein said one or more stabilizing or tonicity agent is selected from the group comprising sodium acetate, sodium bicarbonate, sodium carbonate, sodium chloride, potassium acetate, potassium bicarbonate, potassium carbonate, potassium chloride, calcium chloride, sucrose, glutamate, mannitol, polyols, Polysorbate 20, Polysorbate 40, Polysorbate 80, Poloxamer, Poloxamer 188, Poloxamer 407, amino acids such as histidine, arginine, glycine, methionine, proline, lysine, glutamic acid, amines, cyclodextrins, β-cyclodextrins, polyvinylpyrrolidone, polyethylene glycol 400, sorbitol, trehalose and EDTA, and wherein said one or more stabilizing or tonicity agent is present within said pharmaceutical formulation at a percentage between about 0.001% and about 10%.

4. The pharmaceutical formulation of any one of the preceding claims wherein said anti-OX40 antagonist antibody or fragment thereof is present within said pharmaceutical formulation at a concentration between about 20 mg/mL and about 80 mg/m L, L-histidine buffer is present within said pharmaceutical formulation at a concentration between about 1 mM and 10 mM, sucrose is present within said pharmaceutical formulation at a percentage between about 1% and about 10% and Polysorbate 80 present within said pharmaceutical formulation at a percentage between about 0.001% and about 0.1%, and wherein said pharmaceutical formulation has pH between about 5.0 and about 7.0.

5. The pharmaceutical formulation of any one of the preceding claims wherein said anti-OX40 antagonist antibody or fragment thereof is present within said pharmaceutical formulation at a concentration between about 40 mg/mL and about 60 mg/m L, L-histidine buffer is present within said pharmaceutical formulation at a concentration between about 3 mM and 7 mM, sucrose is present within said pharmaceutical formulation at a percentage between about 2% and about 6% and Polysorbate 80 present within said pharmaceutical formulation at a percentage between about 0.005% and about 0.05%, and wherein said pharmaceutical formulation has pH between about 5.5 and about 6.5.

6. The pharmaceutical formulation of any one of the preceding claims wherein said anti-OX40 antagonist antibody or fragment thereof is present within said pharmaceutical formulation at a concentration of about 48 mg/m L, L-histidine buffer is present within said pharmaceutical formulation at a concentration of about 5 mM, sucrose is present within said pharmaceutical formulation at a percentage of about 4% and Polysorbate 80 present within said pharmaceutical formulation at a percentage of about 0.01%, and wherein said pharmaceutical formulation has pH of about 6.0.

7. The pharmaceutical formulation of any one of the preceding claims, wherein said pharmaceutical formulation is lyophilized and it is stable at about +5±3° C. for at least 24 months, preferably for at least 48 moths, at about +25±2° C. for at least 12 months, and at about +40±2° C. for at least 3 months.

8. The pharmaceutical formulation of claim 8, wherein said lyophilized formulation is reconstituted and comprises said antibody or antibody fragment thereof at a concentration comprised between about 0.5 mg/mL and bout 200 mg/m L.

9. The pharmaceutical formulation of claim 7 or 8, wherein said lyophilized formulation is reconstituted with water for injection to obtain a reconstituted formulation comprising said antibody or antibody fragment thereof at a concentration comprised between about 100 mg/mL and bout 200 mg/m L.

10. The pharmaceutical formulation of claim 9, wherein said reconstituted formulation is further diluted with a dilution media selected from the group comprising saline solution, glucose solution, dextrose solution, to obtain a diluted formulation comprising said antibody or antibody fragment thereof at a concentration comprised between about 0.5 mg/mL and bout 5 mg/m L.

11. The pharmaceutical formulation of any one of claims 1 to 10, wherein said pharmaceutical formulation is suitable for subcutaneous administration or for intravenous administration.

12. The pharmaceutical formulation of any one of the preceding claims for use in the treatment of an OX40-mediated disorder.

13. The pharmaceutical formulation of any one of the preceding claims for use in the treatment of an OX40-mediated disorder selected from the group comprising atopic dermatitis, rheumatoid arthritis, autoimmune uveitis, multiple sclerosis, lupus (such as systemic lupus erythematosus), ulcerative colitis, scleroderma and graft-versus-host disease (GVHD), scleroderma, hidradenitis, and ulcerative colitis.

14. A method of manufacturing the pharmaceutical formulation of any one of the preceding claims.

15. An article of manufacture comprising the pharmaceutical formulation of any one of the preceding claims.
